(19) United States
(12) Patent Application Publication
(10) Pub. No.: US 2014/0324020 A1
(43) Pub. Date: Oct. 30, 2014

(54) Dual Purpose Advisory Device

(71) Applicant: Novo Nordisk A/S, Bagsvaerd (DK)

(72) Inventor: Mads Schenstroem Stefansen, Copenhagen OE (DK)

(21) Appl. No.: 14/349,748

(22) PCT Filed: Oct. 9, 2012
(86) PCT No.: PCT/EP2012/069942

§ 371 Date: Apr. 4, 2014

Related U.S. Application Data
(60) Provisional application No. 61/547,108, filed on Oct. 14, 2011.

(30) Foreign Application Priority Data
Oct. 11, 2011 (EP) 11184701.8

(51) Int. Cl. A61M 5/315; A61M 5/50

(52) U.S. Cl. CPC A61M 5/31535; A61M 5/5086; A61M 2005/3126
USPC 604/506; 604/189; 604/111

(57) ABSTRACT

A system for optimizing a patient's insulin treatment, comprising a drug delivery device (320) with an identifier representing information for a specific drug contained in a reservoir. An accessory device (310) comprises a blood glucose meter (BGM) and a processor programmed to determine for at least two pre-defined specific drugs a recommendation for an amount of the given drug based on a blood glucose value. The recommendation for a given pre-defined drug can be calculated when the corresponding identifier has been captured with the accessory device in a current mounted state, the recommended dose being calculated based on one or more blood glucose values, one or more patient specific parameters as well as parameters for the identified specific type of drug.

The present invention generally relates to systems and methods allowing a patient to optimize drug administration.

### BACKGROUND OF THE INVENTION

In the disclosure of the present invention reference is mostly made to the treatment of diabetes by delivery of insulin, however, this is only an exemplary field of use for the present invention.

Type 2 diabetes is a progressive disease in which β3-cell function deteriorates. Initiating therapy with oral agents is a reasonable approach to take with most patients, the exception being patients with extreme hyperglycemia (fasting plasma glucose >250 mg/dl). These patients require insulin to lower glucose levels. Otherwise, starting with oral therapy can be very effective, especially in patients with a short duration of diabetes and, thus, relatively adequate β-cell function. However, during the course of the decease many patients sooner or later will need therapy with insulin. When determining whether a patient should be put on insulin therapy, it is helpful to look to the guidelines for glycemic control. The American Diabetes Association (ADA) and American College of Endocrinology (ACE) publish goals for A1C, postprandial glucose, and fasting/pre-prandial glucose. Most patients who are unable to achieve these goals using oral agents are candidates for insulin therapy.

One type of initial insulin therapy for Type 2 diabetics is to use once-daily injections with a long-acting basal insulin such as Levemir® from Novo Nordisk, often in combination with oral antidiabetic agents. However, to be successful, insulin therapy requires timely and appropriate titration of dosages. For example, in combination with oral antidiabetic agents it is recommended to initiate Levemir® treatment with once daily administration at a dose of 10 U or 0.1-0.2 U/kg. The dose of Levemir® should then be titrated based on individual patients' needs, e.g. based on average (3-7 measurements) self-measured pre-breakfast BG values. For example, for a calculated value of >10.0 mmol/L it is recommended to adjust the Levemir® dose with +8 units, for a calculated value of 9.1-10.0 mmol/L it is recommended to adjust the Levemir® dose with +6 units, for a calculated value of 8.1-9.0 mmol/L it is recommended to adjust the Levemir® dose with +4 units, for a calculated value of 7.1-8.0 mmol/L it is recommended to adjust the Levemir® dose with +2 units, and for a calculated value of 6.1-7.0 mmol/L it is recommended to adjust the Levemir® dose with +2 units. If one BG measurement is 3.1-4.0 mmol/L it is recommended to adjust the Levemir® dose with −2 units, and if one BG measurement is <3.1 mmol/L it is recommended to adjust the Levemir® dose with −4 units. The calculation of the average pre-breakfast BG values as well as the resulting Levemir® dose adjustments may either be performed by the patient him/herself or by a doctor/nurse based on BG values supplied by the patient. As appears, such a regimen is both time-consuming as well as prone to mistakes. This said, self-titration regimens are considered to facilitate empowerment of patients, allowing them to become more involved in their treatment which may then result in improved glycaemic control.

Correspondingly, devices and systems have been provided in which recommendations are generated based on self-measured BG values by a pre-programmed algorithm, e.g. corresponding to the relatively simple titration regimen described above. Indeed, much more sophisticated algorithms can be implemented taking into account e.g. patient characteristics and other variable inputs, see e.g. US 2009/0253970 hereby incorporated by reference. The algorithm may be in the form of software adapted to run on different platforms, e.g. PC, PDA or smartphone, or it may be imbedded in a device such as a blood glucose meter, BGM, see e.g. US 2010/0016700 hereby incorporated by reference.

Although the above disclosure addresses the specific issue of titration of a dose of basal insulin for a patient introduced on insulin, it may also be beneficial from time to time to recalculate the basal dose after the initial titration regimen has ended.

A further aspect in which patient treatment can be supported by calculated recommendations is when diabetic patients are in need of meal related injections of insulin, i.e. bolus treatment with fast-acting insulin for which a bolus calculator may help the patient. For example, a given bolus calculator may calculate bolus recommendations based on the variables current BG and total grams of carbohydrate (CHO) in the planned meal, as well as patient-specific settings for target BG, carbohydrate-to-insulin ratios (CIR), and insulin sensitivity factor (ISF).

Systems have been proposed which are able to calculate both basal insulin doses and bolus insulin doses based on a number of parameters. For example, US 2008/0262469 discloses a system adapted to receive BG information from a continuous blood glucose monitor (CGM) as well as information in respect of administered amounts of basal and/or bolus insulin. Based on this as well as further information the system is adapted to provide suggestions for both basal and bolus treatment. In specific embodiments the insulin dose information may be transmitted wirelessly from individual drug delivery devices of the pen type.

Although such automatically generated recommendations may be of great help to both medical staff and patients, the recommendations may be wrong if they are misunderstood by the patient, e.g. when a basal dose is confused with a bolus dose.

Having regard to the above, it is an object of the present invention to provide systems and methods supporting cost-effective and safe optimization and control of treatment for diabetic patients.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, in a first aspect of the invention a system for optimizing a patient's insulin treatment is provided, comprising a drug delivery device comprising a reservoir containing a drug, a drug expelling mechanism for expelling drug from the reservoir, and an identifier representing information for the specific drug contained in the reservoir, as well as an accessory device releasably mountable on the drug delivery device corresponding to a mounted state. The accessory device comprises a blood glucose meter for receiving a patient blood sample and generating a blood glucose value for the patient blood sample, memory means adapted to receive and store blood glucose values as well as a patient specific parameters related to insulin treatment, means for capturing drug information from the identifier, output means (e.g. a LCD or OLED) for communicating to the patient a recommended dose of drug, and input means allowing a user to input patient specific parameters. The accessory device further comprises a processor operatively connected to the memory means, the processor comprising program instructions to determine for at least two pre-defined specific drugs a recommendation for an amount of the given drug. The recommendation for a given pre-defined specific drug can be calculated when the corresponding identifier has been captured with the accessory device in a current mounted state, the recommended dose being calculated based on one or more blood glucose values, one or more patient specific parameters as well as parameters for the identified specific type of drug.

In a further aspect of the invention an accessory device as defined above is provided per se, the accessory device being adapted to be mounted on a drug delivery as defined above.

By these arrangements it is assured with a high certainty that a recommendation is based on the type of insulin contained in the drug delivery device in combination with which the accessory device is actually used. The current mounted state is the state since the accessory device was mounted for the last time, the accessory device comprising means to detect when it is mounted and de-mounted. In the mounted position the accessory device may be locked e.g. axially and/or rotationally, this creating a pre-defined mounted (or mated) condition. If no identifier can be captured during a given mounting state the patient may be informed and no recommendation be calculated.

The identifier information being may be captured just prior to calculation of a recommended dose of drug or at an earlier point in time during the current mounting state. The accessory device could be configured e.g. as a pen cap or as a device adapted to be mounted on or around the body of a drug delivery device.

The identifier may be of any suitable type allowing information to be captured. It may be associated with the reservoir or for a pre-filled drug delivery device also with other parts of the device. The information may be captured by e.g. optical, mechanical or electronic means. The identifier could be a simple identifier providing information about e.g. “type A drug” and “type B drug” where the relevant parameters for the drug A and B would then be known by the system. Alternatively, the relevant parameters, e.g. drug uptake profile and strength, could be comprised in a code. Other relevant parameters could be e.g. manufacturing date preventing use of too old drugs.

In a specific embodiment one of the pre-defined drugs is a basal, long-acting insulin such as Levemir®, the processor comprising program instructions to determine for the given drug and from the blood glucose values determined at a plurality of times whether and by how much to vary a patient's presently stored basal insulin dosage regimen in order to maintain the patient's future blood-glucose-level measurements within a predefined range. Such functionality may be used as an initial titration aid or as a more long-term aid for a basal insulin regimen.

In a further specific embodiment one of the pre-defined drugs is a bolus, fast-acting insulin such as NovoLog®/NovoRapid®, the processor comprising program instructions to determine for the given drug, from a blood glucose value and a carbohydrate value input by the user, an amount of bolus insulin to be taken in order to maintain the patient's future blood-glucose-level measurements within a predefined range. In order to allow a user to use the bolus calculator based only on a carbohydrate value for a planned meal, the user may be able to control the processor to calculate an amount of bolus insulin using a pre-set blood glucose value, e.g. an average pre-meal value for the given patient.

In a further aspect of the invention a method for optimizing a patient's insulin treatment is provided, comprising the steps of providing (i) one or more drug delivery devices each comprising a reservoir containing a drug, a drug expelling mechanism for expelling drug from the reservoir, and an identifier representing information for the specific drug contained in the reservoir, and (ii) an accessory device releasably mountable on the drug delivery device corresponding to a mounted state, the accessory device comprising a blood glucose meter for receiving a patient blood sample and generating a blood glucose value for the patient blood sample, memory means adapted to receive and store blood glucose values as well as patient specific parameters related to insulin treatment, means for capturing drug information from the identifier, output means for communicating to the patient a recommended dose of drug, input means allowing a user to input patient specific parameters, and a processor operatively connected to the memory means, the processor comprising program instructions to calculate for at least two pre-defined specific drugs a recommendation for an amount of the given drug. The method comprises the further steps of capturing an identifier, detecting that the accessory device is in a mounted state, and calculating a recommendation for a given pre-defined specific drug when the corresponding identifier has been captured with the accessory device in the current mounted state, the recommended dose being calculated based on one or more blood glucose values, one or more patient specific parameters as well as parameters for the identified specific type of drug. The provided devices may be modified as described above.

In a further aspect of the invention a system for optimizing a patient's insulin treatment is provided, comprising a drug reservoir or means for receiving a drug reservoir, setting means allowing a patient to set a dose to be expelled from the drug reservoir, a drug expelling mechanism for expelling a set dose of drug from the reservoir, and a blood glucose meter for receiving a patient blood sample and generate a blood glucose value based on the patient blood sample. The system further comprises a processor adapted to generate a suggestion for a dose of drug to take in order to keep the patient's future blood-glucose-level within a pre-set range, and display means controlled by the processor, wherein the display is controlled to display generated suggestions but not blood glucose values. In case the processor generates a recommendation for a basal dose, the user may be prompted to indicate whether a fasting or non-fasting BG measurement is made. By this arrangement it is provided that (i) a BG value cannot be confused with a recommended dose, and (ii) that users who do not bother with actual BG values but are only interested in the recommendation do not have to consider a BG value. If a given BG value is outside a pre-defined range, e.g. because of an error in the procedures, this may be indicated simply as “error” and the user may be prompted to repeat the procedure. If the BG value is still outside the pre-set range, the user may be prompted to consult his/her doctor or the processor may be provided with an algorithm designed to take into account such events, e.g. by ignoring such a value and base e.g. a basal does recommendation on previously determined and stored BG values. This said, the system may be designed to allow a BG value to be displayed when specifically required by the user, e.g. by a sequence of button actuations.

In a further aspect of the invention a system for optimizing a patient's insulin treatment is provided, comprising a BGM for receiving a patient blood sample and generating a blood glucose value of the patient blood sample, a processor adapted to generate a suggestion for a dose of drug to take in order to keep the patient's future blood-glucose-level within a predefined range, and display means for communicating blood glucose values and suggested dose values to the patient, wherein the display is controlled to (i) display blood glucose values in accordance with a first visual mode, and (ii) display suggested dose values in accordance with a second visual mode. By this feature the risk that a BG value is confused with a recommended dose is reduced.

In an exemplary embodiment the displayed values are formed by segments on a background, the segments being controllable between a first and a second visual condition and the background being controllable between a first and a second visual condition, wherein BG values are displayed by segments in their first visual condition on a background in its second visual condition, and dose values are displayed by segments in their second visual condition on a background in its first visual condition. The display may be of the dot-matrix type, the dots forming the segments as well as the background, each dot thus being adapted to generate the first and second visual condition for both segments and background. In this way a high contrast between the two different showings is provided in a cost-effective way. The BG values and the dose values may be displayed on the same area of the display, e.g. in case a small display is used.

In a specific embodiment the system may be in the form of a drug delivery assembly comprising a drug delivery device comprising the drug reservoir or means for receiving a drug reservoir, the setting means, and the drug expelling mechanism, as well as an add-on device releasably mountable on the drug delivery device, comprising the BGM, the processor, and the display means.

In a yet further aspect of the invention a system for optimizing a patient's insulin treatment is provided, comprising a drug reservoir or means for receiving a drug reservoir, setting means allowing a patient to set a dose to be expelled from the drug reservoir, a drug expelling mechanism for expelling a set dose of drug from the reservoir, and a BGM for receiving a patient blood sample and generate a blood glucose value based on the patient blood sample. The system further comprises a processor adapted to generate a suggestion for a dose of drug to take in order to keep the patient's future blood-glucose-level within a predefined range, and display means controlled by the processor to display generated dose suggestions, detecting means for detecting when a patient-actuated indicator operation is performed, the indicator operation being indicative of the patient's intend to administer a dose of drug, and timer means for determining a period of time between two patient-actuated indicator operations, wherein an alarm is actuated when the period of time between two patient-actuated indicator operations is less than a pre-set period of time. For example, when the system is adapted to calculate a basal insulin dose to be taken once daily, then an alarm, e.g. visual or audible, may be actuated if the user intends to take a dose of insulin within e.g. 18 hours. If the user makes a second BG measurement within the pre-set period of time, the alarm may comprise to control the display means to display a visual warning and not to display a generated dose suggestion. If the user is certain that a previous patient-actuated indicator operation did not result in administering a dose of drug, patient input means may allow a patient to prompt the system to display the last generated dose suggestion.

In a specific embodiment the system may be in the form of a drug delivery assembly comprising a drug delivery device comprising the drug reservoir or means for receiving a drug reservoir, the setting means, and the drug expelling mechanism, as well as a cap device releasably mountable on the drug delivery device, comprising the BGM, the processor, the display means, and the detecting means. In such a system the cap device may further comprises means to detect a cap-off event when the cap device has been at least partially demounted from the drug delivery device for a pre-determined amount of time, the indicator operation being the detection of a cap-off event.

In a further aspect of the invention a system for optimizing a patient's insulin treatment is provided, comprising a drug reservoir or means for receiving a drug reservoir, first setting means allowing a patient to set a dose to be expelled from the drug reservoir, and a drug expelling mechanism for expelling a set dose of drug from the reservoir, a blood glucose meter for receiving a patient blood sample and generating a blood glucose value of the patient blood sample, and a processor adapted to generate a suggestion for a dose of drug to take in order to keep the patient's future blood-glucose-level within a predefined range. In order to secure that the calculated dose is set correctly, the system further comprises second setting means controlled by the processor for setting a dose to be expelled from the drug reservoir corresponding to the generated suggestion. The system may comprise output means for communicating to the patient the generated suggestion.

The first setting means may be mechanical and comprise a dose setting element adapted to be manipulated by the patient, and the second setting means may correspondingly adapted to also manipulate the dose setting element. Such a system may be in the form of a drug delivery assembly comprising a drug delivery device comprising the drug reservoir or means for receiving a drug reservoir, the first setting means, and the drug expelling mechanism, as well as an add-on device releasably mountable on the drug delivery device, comprising the BGM, the processor, and the second setting mean.

As used herein, the term “insulin” is meant to encompass any drug-containing flowable medicine capable of being passed through a delivery means such as a cannula or hollow needle in a controlled manner, such as a liquid, solution, gel or fine suspension, and which has a blood glucose controlling effect, e.g. human insulin and analogues thereof as well as non-insulins such as GLP-1 and analogues thereof. In the description of the exemplary embodiments reference will be made to the use of insulin. Correspondingly, the term “subcutaneous” infusion is meant to encompass any method of transcutaneous delivery to a subject.

### BRIEF DESCRIPTION OF THE DRAWINGS

In the following the invention will be further described with reference to the drawings, wherein

FIG. 1 shows a drug delivery device in combination with a mounted cap device,

FIG. 2 shows a further cap device,

FIG. 3 shows an exploded view of the cap device of FIG. 2,

FIG. 4 shows a further cap device mounted on a drug delivery device,

FIG. 5 shows a drug delivery device comprising electronic logging and display means,

FIG. 6 shows a cap device in combination with two drug delivery devices, and

FIGS. 7A-7E show different alternatives for controlling a display.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only.

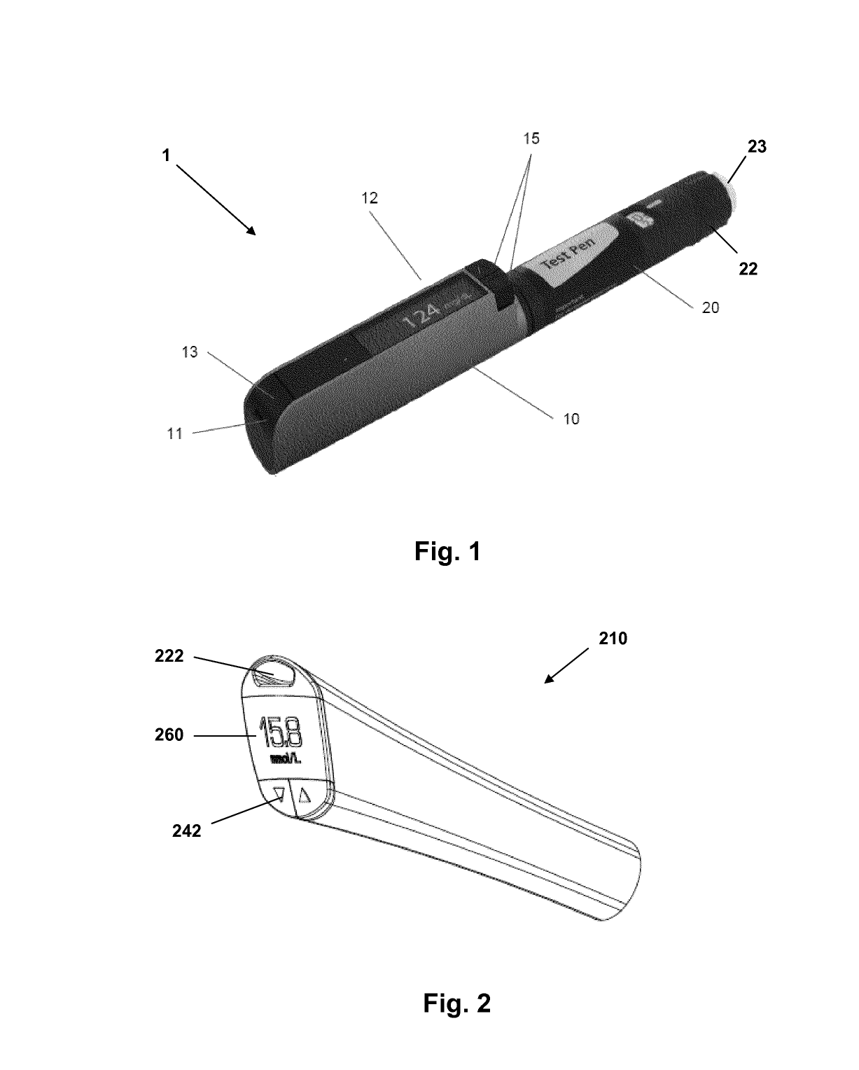

FIG. 1 shows a drug delivery assembly 1 comprising a pre-filled drug delivery device 20 onto which an accessory device in the form of a cap device 10 is mounted. The drug delivery device comprises a reservoir containing a drug, a drug expelling mechanism for expelling drug from the reservoir, a rotatable dose setting member 22 and a release button 23, as well as an identifier representing information for the specific drug contained in the reservoir. The identifier may be of any suitable type allowing information to be captured. It may be associated with the reservoir or for a pre-filled drug delivery device as shown also with other parts of the device. The information may be configured to be captured by e.g. optical, mechanical or electronic means. The identifier could be a simple identifier allowing a pre-determined type of drug to be recognized with the relevant parameters for the drug then be known by the system. Alternatively, the relevant parameters, e.g. drug uptake profile and strength, could be comprised in a code. The identifier may be unique for a given reservoir or device.

The cap comprises a strip port for a BGM arranged in the interior of the cap and configured for receiving a patient blood sample on a strip and generating a blood glucose value for the patient blood sample, a display 12 adapted to show BG and other values and recommendations, an input button 13 adapted to among other functions confirm a given value, e.g. a BG reading and toggle between different modes, as well as a set of up/down buttons 15 adapted to scroll in a given log, e.g. a BG log, or input a value, e.g. a carbohydrate amount for a planned meal. The cap also comprises means for detecting when it has been removed from and/or attached to the drug delivery device.

The cap device further comprises memory means adapted to receive and store blood glucose values as well as a patient specific parameters related to insulin treatment, e.g. target BG, carbohydrate-to-insulin ratios (CIR), and insulin sensitivity factor (ISF), and means for capturing drug information from the identifier, the capturing means corresponding to the type of identifier actually used.

To generate recommendations the cap device comprises a processor operatively connected to the memory means, the processor comprising program instructions to determine for at least two pre-defined specific drugs a recommendation for an amount of the given drug. In an exemplary embodiment of the system a recommendation for a given pre-defined specific drug can be calculated when the system has detected that the cap device is mounted on a corresponding drug delivery device and via the identifier detected and recognized the drug contained in the reservoir of the drug delivery device, i.e. two conditions have to be fulfilled. The identifier may be captured just prior to the calculation of a recommendation or, alternatively, when the cap is mounted. The cap may compare the current identifier with the previously captured identifier and inform a user correspondingly, i.e. that a different drug is detected or a new cartridge containing the same drug. To further assure that a user will not use the cap device for a wrong type of drug, the cap device may be functional (e.g. the display turned on) only when mounted on a corresponding drug delivery device comprising a recognized identifier. Thus with the cap device in its mounted position and a recommendation shown on the display, the user can set and subsequently expel a desired dose, e.g. corresponding to the recommended dose size.

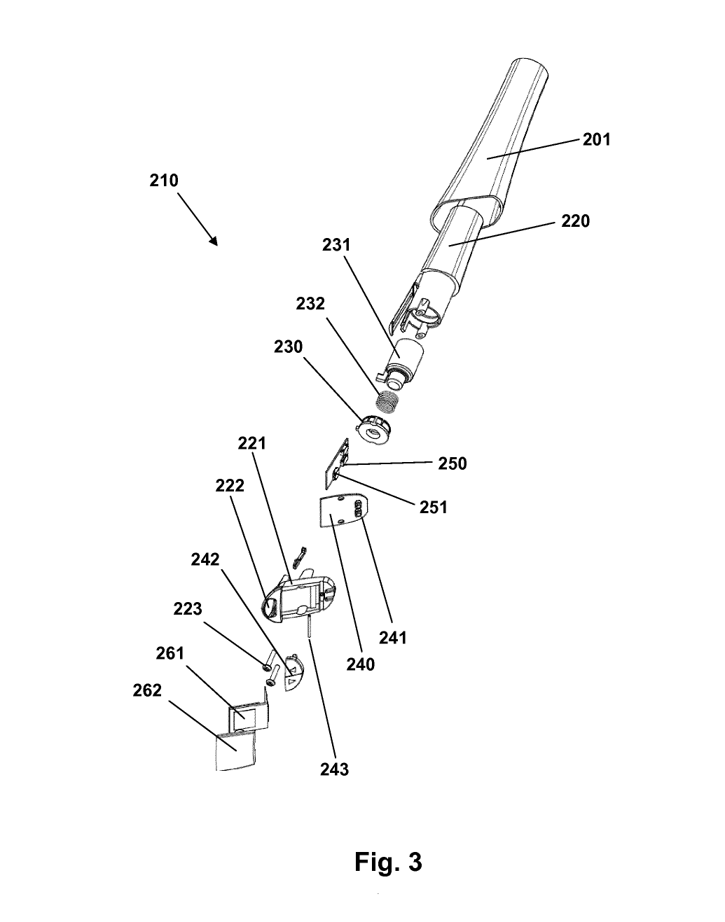

FIG. 2 shows an alternative configuration of a BGM cap 210 in which a strip port 222, a display 260 and user buttons 242 are arranged at the distal end of the cap. As seen in FIG. 3 the cap 210 comprises a housing member 201 in which a generally tubular main chassis 220 is arranged, the latter having a top chassis 221 with a strip port 222 attached by screws 223. A spring support member 230 is attached to the main chassis. An actuator cup 231 is slidingly received in the main chassis in which it can travel between an un-loaded and a loaded position, the cup being biased towards its initial position by a spring 232 arranged between the spring support and the distal end of the cup, the cup being adapted to be moved distally when the cap is mounted on the distal end of a corresponding drug delivery device as seen in FIG. 1. The cap further comprises a first PCB 240 on which button switches 241 as well as an energy source and processor and memory means (not seen) are arranged, and a second PCB 250 on which cup-actuated switch means 251 and a BGM unit (not shown) are arranged. Provided in the interior of the cap and connected to the processor is arranged detections means (not shown) for a reservoir identifier. The switch means 251 is actuated when the cup 231 is moved axially thereby detecting a cap-off or cap-on event. A display 261 is connected to the first PCB and covered by a transparent window member 262 attached to the top chassis to which further is attached two buttons 242 by means of an axel 243 allowing the buttons to pivot.

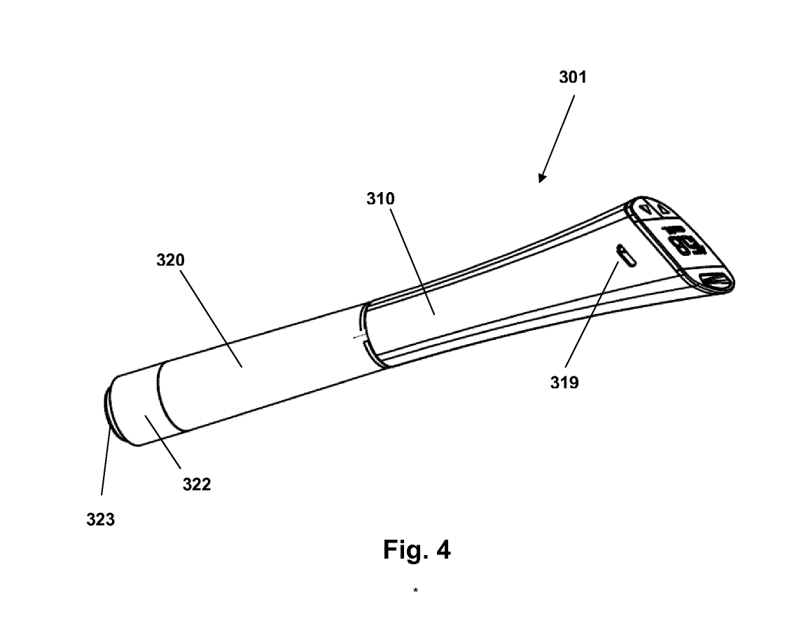

FIG. 4 shows a drug delivery assembly 301 comprising a drug delivery device 320 of the type shown in FIG. 1 and thus comprising a rotatable dose setting member 322 and a release button 323, and onto which is mounted a cap device 310. The cap device is similar to the BGM cap device of FIG. 2, however, the cap device is provided with an I/O port 319 adapted for wired communication, e.g. allowing entering of values and update of software. Alternatively, the cap device may be provided with wireless communication means.

In the shown embodiment of FIG. 1 the drug delivery device is a pre-filled pen device intended for single use only, however, the pen could also be a durable device intended to be used with exchangeable drug cartridges. In case the pen is of the durable type it may be provided with electronic means for detecting and creating a dose log as well as display means.

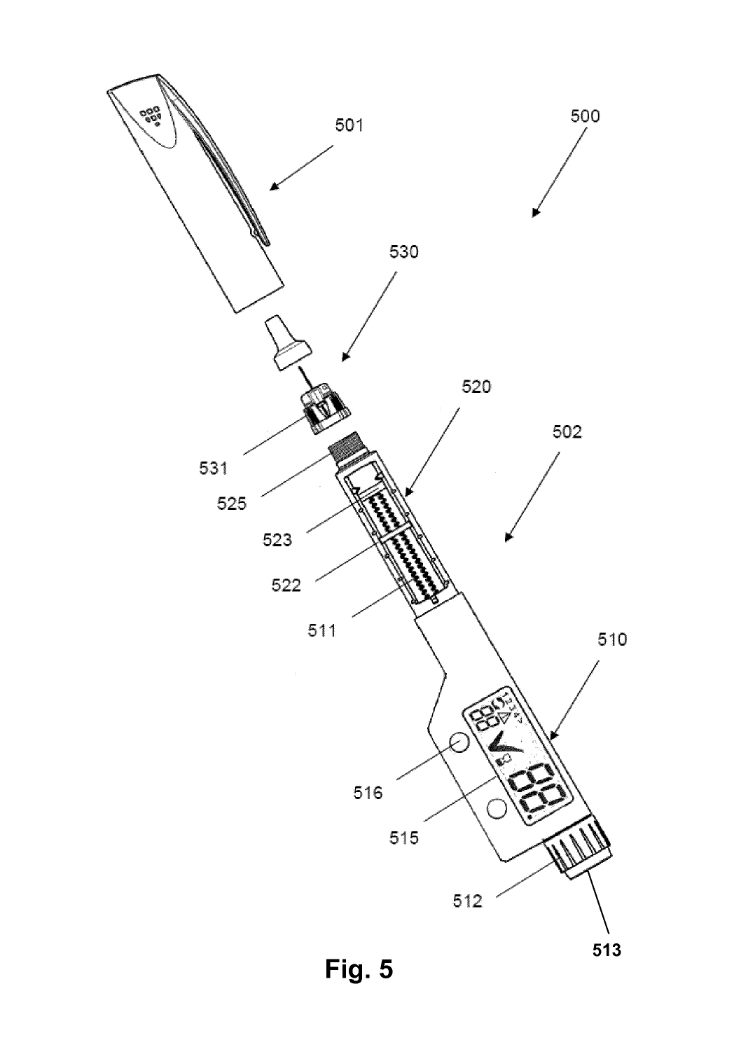

FIG. 5 shows such a pen 500. The pen device comprises a cap portion 501 (here shown as a normal cap not related to the cap device 10) and a main portion 502 having a proximal part 510 in which a drug expelling mechanism is arranged, and a distal reservoir part 520 in which a replaceable drug-filled transparent cartridge 521 with a distal needle-penetratable septum is arranged and hold in place by a cartridge holder 522 releasably mounted to the proximal part, the cartridge holder having openings allowing a portion of the cartridge to be inspected. The cartridge is provided with a piston 523 driven by a piston rod 511 forming part of the expelling mechanism, the piston rod being adapted to be pushed back when a new cartridge is mounted. A proximal-most rotatable ring member 512 serves to manually set a desired dose of drug which can then be expelled when the release button 513 is actuated. This type of a mechanical pen-formed drug delivery device is well known, see e.g. WO 99/38554 to which reference is made for further details in respect of the internal construction of the shown type of pen. The cartridge (or alternatively the cartridge holder) is provided with distal coupling means in the form of a hub mount 525 having, in the shown example, an external thread adapted to engage an inner thread of a hub 531 of a needle assembly 530. The proximal part further comprises a display 515, user actuatable keys 516 as well as electronic means (not shown) for detecting and storing information representing operations performed by the expelling mechanism. Also the pen may be adapted to read information from a reservoir identifier.

The detection means for detecting a set and/or expelled dose may be adapted to detect directly or indirectly the position of the piston rod, see e.g. U.S. Pat. No. 6,585,698 which is hereby incorporated by reference. The electronic means is adapted to store data representing injections performed by the user in the form of a time and dose log. The display may show the actual dose being set by a user using the dose setting member 512, the last dose (e.g. amounts of units expelled) and the time since last dose (or the actual time for the last dose), or the user may use the keys 516 to scroll through the log to display previous expelling data. In case the drug delivery device is in the form of an electronically controlled motorized device, the recommended dose may be automatically transferred to the delivery device such that a user just has to accept the set dose.

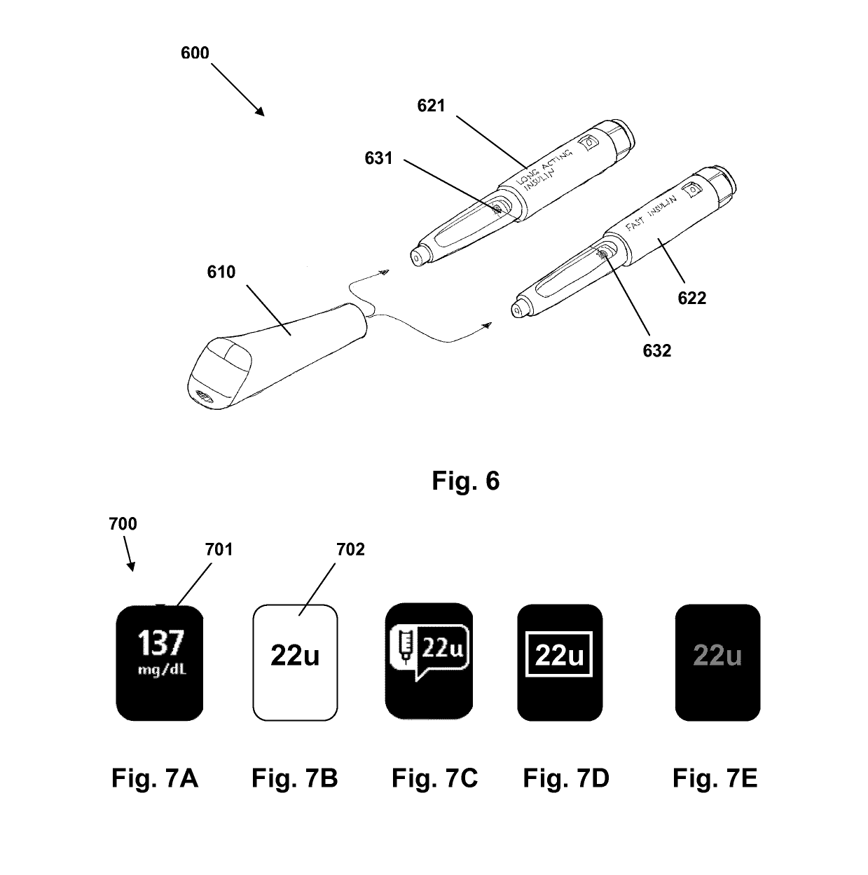

FIG. 6 shows a system 600 comprising a dose recommendation cap device 610 as described with reference to FIG. 1 in combination with first and second pen-formed drug delivery devices 621, 622 of the type shown also in FIG. 1. The first drug delivery device 621 comprises a reservoir containing a long acting insulin formulation as well as an identifier in the form of a first RFID tag 631, this providing that the cap device serves as a basal titrator when mounted on the first drug delivery device. The second drug delivery device 622 comprises a reservoir containing a fast acting insulin formulation as well as an identifier in the form of a second RFID tag 632, this providing that the cap device serves as a bolus calculator when mounted on the second drug delivery device.

With reference to FIGS. 1-6 an accessory device in the form of a cap device 10 is mounted, however, the accessory device may also have other configurations. For example, it may be in the form of a body mounted device which does not have to be removed when a dose of drug is expelled as shown in e.g. WO 2010/037828.

On the above-described cap devices and other devices that can display a measurement and give a recommendation, e.g. on an optimal dose of drug, it is important that the user clearly can differentiate between measurements and a recommendations. If the area for displaying the information is relatively small is it not possible to use large icons or easily readable text.

FIGS. 7A-7E show a dot-matrix display 700 of the same configuration and size as the displays shown in the FIGS. 2-4 embodiments, the display being controlled to enhance the visual differentiation when showing different types of values. The biggest possible contrast between e.g. measurements 701 and an advice 702 is to invert the display. If the normal information is white text on a black background (see FIG. 7A) then black text on a white background is a significant change (see FIG. 7B). An alternative could be to show the recommendation in a call-out (see FIG. 7C) or a frame around the recommendation (see FIG. 7D). Alternatively a different text colour could be used (see FIG. 7E) if the device is equipped with a colour display. The alternatives could be combined just as they could be supplemented with a relevant icon.

In the above description of a preferred embodiment, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A system for optimizing a patient's insulin treatment, comprising:
a drug delivery device comprising:
a reservoir containing a drug,
a drug expelling mechanism for expelling drug from the reservoir, and
an identifier representing information for the specific drug contained in the reservoir,
an accessory device releasably mountable on the drug delivery device corresponding to a mounted state, the accessory device comprising:
a blood glucose meter for receiving a patient blood sample and generating a blood glucose value for the patient blood sample,
memory structure adapted to receive and store blood glucose values as well as patient specific parameters related to insulin treatment,
structure for capturing drug information from the identifier,
output structure for communicating to the patient a recommended dose of drug,
input structure allowing a user to input patient specific parameters,
a processor operatively connected to the memory structure, the processor comprising program instructions to calculate for at least two pre-defined specific drugs a recommendation for an amount of the given drug,
wherein a recommendation for a given pre-defined specific drug can be calculated when the corresponding identifier has been captured with the accessory device in the current mounted state, and
wherein the recommended dose is calculated based on one or more blood glucose values, one or more patient specific parameters as well as parameters for the identified specific type of drug.

2. A system as in claim 1, wherein one of the pre-defined drugs is a basal insulin, the processor comprising program instructions to calculate for the given drug and from the blood glucose values determined at a plurality of times whether and by how much to vary a patient's presently stored basal insulin dosage regimen in order to maintain the patient's future blood-glucose-level measurements within a predefined range.

3. A system as in claim 1, wherein one of the pre-defined drugs is a bolus insulin, the processor comprising program instructions to calculate for the given drug, from a blood glucose value and a carbohydrate value input by the user, an amount of bolus insulin to be taken in order to maintain the patient's future blood-glucose-level measurements within a predefined range.

4. A system as in claim 3, wherein the user can control the processor to calculate an amount of bolus insulin using a pre-set blood glucose value.

5. A system as in claim 1, the accessory device further comprising:
detecting structure for detecting when a patient-actuated indicator operation is performed, the indicator operation being indicative of the patient's intend to administer a dose of drug,
timer structure for determining a period of time between two patient-actuated indicator operations,
wherein an alarm is actuated when the period of time between two patient-actuated indicator operations is less than a pre-set period of time.

6. A system as in claim 5, wherein a first pre-set period of time is used when the identified drug is a bolus insulin, and a second pre-set period of time is used when the identified drug is a basal insulin.

7. A system as in claim 5, wherein the accessory device further comprises structure to detect a cap-off event when the accessory device has been at least partially de-mounted from the drug delivery device for a predetermined amount of time, and the indicator operation is the detection of a cap-off event.

8. A system as in claim 5, wherein the display is controlled to display calculated suggestions but not blood glucose values.

9. A system as in claim 5, wherein the output structure is in the form of a display controlled to display blood glucose values in accordance with a first visual mode, and display calculated dose values in accordance with a second visual mode.

10. A system as in claim 9, wherein displayed values are formed by segments on a background, the segments being controllable between a first and a second visual condition and the background being controllable between a first and a second visual condition, blood glucose values being displayed by segments in their first visual condition on a background in its second visual condition, and dose values being displayed by segments in their second visual condition on a background in its first visual condition.

11. A system as in claim 10, wherein the display is of the dot-matrix type, the dots forming the segments as well as the background, each dot thus being adapted to generate the first and second visual condition for both segments and background.

12. A system as in claim 9, wherein the accessory device is adapted to transmit stored data to an external device or system.

13. A system as in any of the previous claims claim 9, wherein the accessory device is in the form of a cap device adapted to be mounted to cover a reservoir outlet.

14. A device for optimizing a patient's insulin treatment and adapted to be releasably mountable on a drug delivery device corresponding to a mounted state, the drug delivery device comprising:
a reservoir containing a drug,
a drug expelling mechanism for expelling drug from the reservoir, and
an identifier representing information for the specific drug contained in the reservoir, the accessory device comprising:
a blood glucose meter for receiving a patient blood sample and generating a blood glucose value for the patient blood sample,
memory structure adapted to receive and store blood glucose values as well as patient specific parameters related to insulin treatment,
structure for capturing drug information from the identifier,
output structure for communicating to the patient a recommended dose of drug,
input structure allowing a user to input patient specific parameters,
a processor operatively connected to the memory structure, the processor comprising program instructions to calculate for at least two pre-defined specific drugs a recommendation for an amount of the given drug,
wherein a recommendation for a given pre-defined specific drug can be calculated when the corresponding identifier has been captured with the accessory device in a current mounted state, and
wherein the recommended dose is calculated based on one or more blood glucose values, one or more patient specific parameters as well as parameters for the identified specific type of drug.

15. A method for optimizing a patient's insulin treatment, comprising the steps of:
providing one or more drug delivery devices each comprising:
a reservoir containing a drug,
a drug expelling mechanism for expelling drug from the reservoir, and
an identifier representing information for the specific drug contained in the reservoir,
providing an accessory device releasably mountable on the drug delivery device corresponding to a mounted state, the accessory device comprising:
a blood glucose meter for receiving a patient blood sample and generating a blood glucose value for the patient blood sample,
memory structure adapted to receive and store blood glucose values as well as patient specific parameters related to insulin treatment,
structure for capturing drug information from the identifier,
output structure for communicating to the patient a recommended dose of drug,
input structure allowing a user to input patient specific parameters,
a processor operatively connected to the memory structure, the processor comprising program instructions to calculate for at least two pre-defined specific drugs a recommendation for an amount of the given drug,
capturing an identifier,
detecting that the accessory device is in a mounted state,
calculating a recommendation for a given pre-defined specific drug when the corresponding identifier has been captured with the accessory device in the current mounted state, the recommended dose being calculated based on one or more blood glucose values, one or more patient specific parameters as well as parameters for the identified specific type of drug.
